(19) United States
(12) Patent Application Publication
(10) Pub. No.: US 2009/0198142 A1
(43) Pub. Date: Aug. 6, 2009

(54) HEART PARAMETER MONITOR

(76) Inventors: Jon R. Stabler, San Antonio, TX (US); William H. Righter, Portland, OR (US)

Correspondence Address: KOLISCH HARTWELL, P.C., 200 PACIFIC BUILDING, 520 SW YAMHILL STREET, PORTLAND, OR, 97204, US

(21) Appl. No.: 12/397,109

(22) Filed: Mar. 3, 2009

Related U.S. Application Data
(63) Continuation of application No. 11/410,567, filed on Apr. 24, 2006, now abandoned.
Application No. 11/410,567 is a continuation of application No. 11/236,433, filed on Sep. 26, 2005, now abandoned.
Application No. 11/236,433 is a continuation of application No. 10/861,106, filed on Jun. 3, 2004.
(60) Provisional application No. 60/475,798, filed on Jun. 3, 2003.

(51) Int. Cl. A61B 5/02

(52) U.S. Cl. 600/500

(57) ABSTRACT

An electronic device to measure the level of mental activity of an individual includes measurement structure for measuring the interval between every heart beat of the individual. The device also includes analysis structure that is constructed to perform a series of mathematical calculations ultimately to compute an index number for the individual. That index number is a measure of mental activity of the individual and is usable to predict the quality of the individual's performance in a desired activity.

### CROSS-REFERENCE TO RELATED APPLICATIONS

This application is a continuation of U.S. patent application Ser. No. 11/410,567, filed Apr. 24, 2006 and entitled “Heart Parameter Monitor”, which application is a continuation of U.S. patent application Ser. No. 11/236,433, filed Sep. 26, 2005 and entitled “Heart Parameter Monitor”, which application claims priority to U.S. patent application Ser. No. 10/861,106, filed Jun. 3, 2004 and entitled “Heart Parameter Monitor”, which application claims priority to U.S. Provisional Patent Application Ser. No. 60/475,798, filed Jun. 3, 2003, and entitled “Heart Rate Monitor”.

### SUMMARY

A simplified memory-micro structure is possible for the Heart Parameter Monitor invention using serial flash with a small footprint micro, cutting digital real estate in half. An ST M25P10 coupled to an NEC uPD 789417 or 789881. Coupled with a pair of Maxim quad op-amps, we have a complete electronics package. The monitor can be used to measure desired, usable heart parameters such as heart rate.

### BRIEF DESCRIPTION OF THE DRAWINGS

FIGS. 1-4 are schematic diagrams showing analog circuitry to determine Heart Rate Variability (HRV) according to the invention.

FIGS. 5-6 are schematic diagrams showing digital circuitry to determine HRV according to the invention.

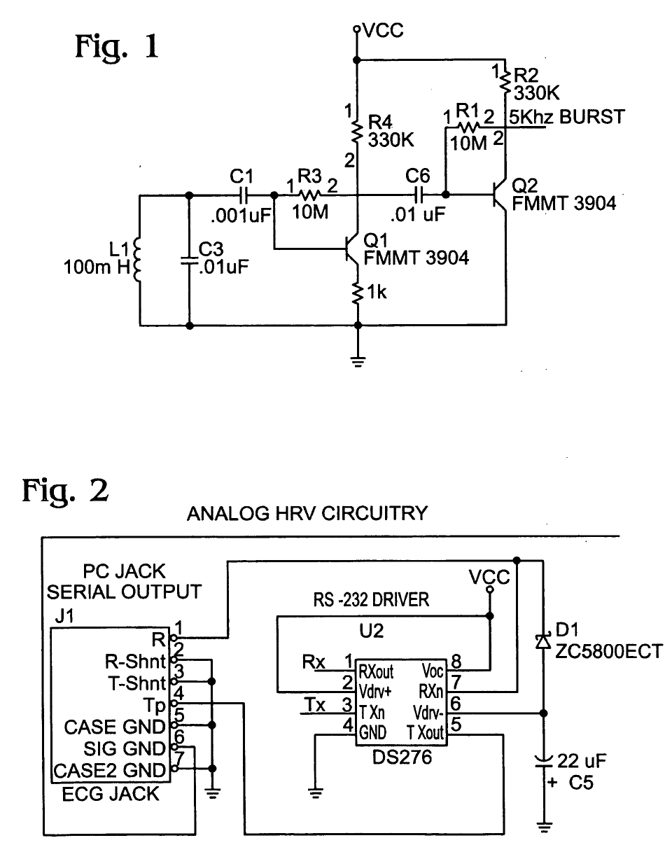

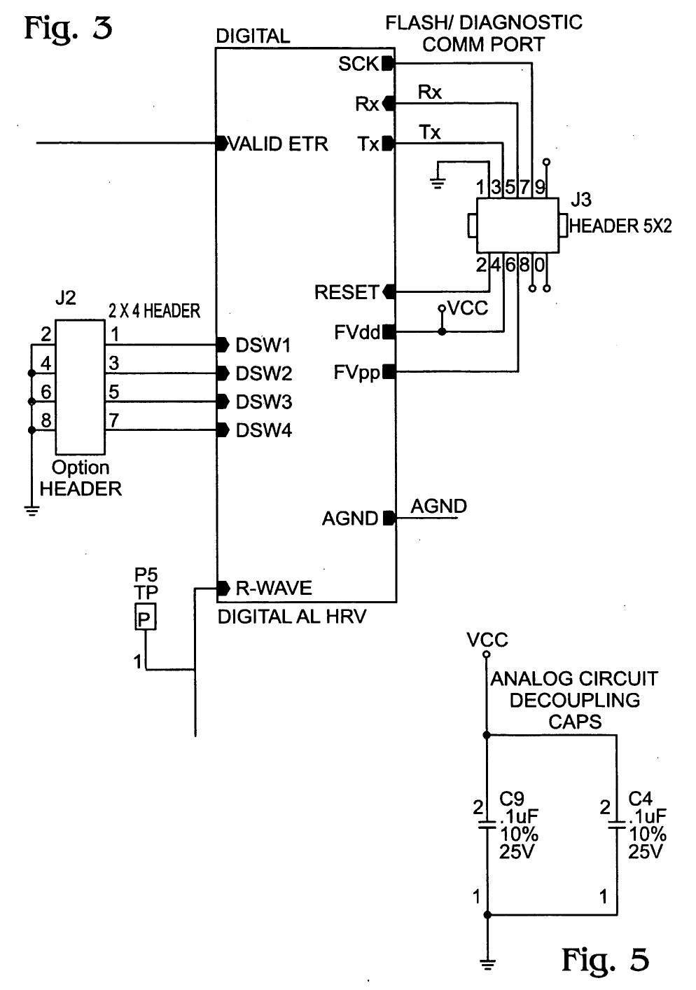

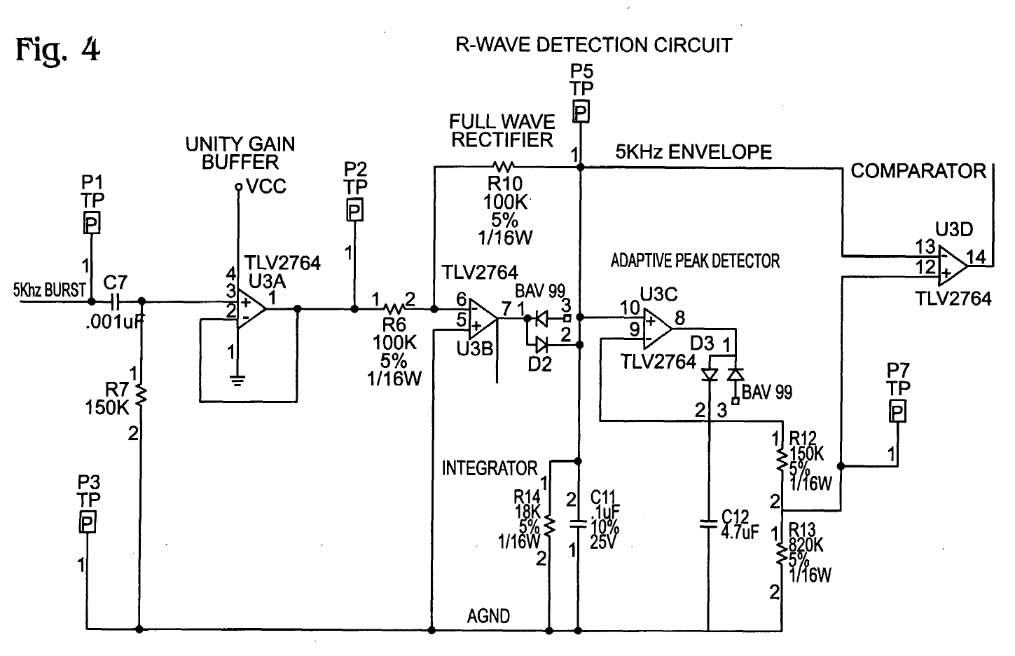

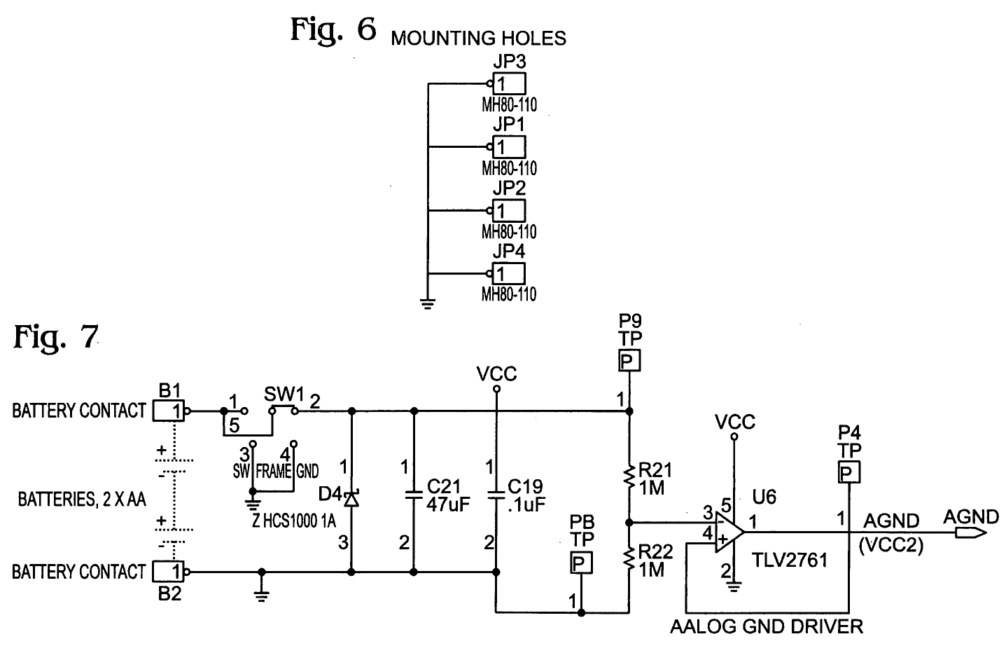

### TECHNICAL DESCRIPTION OF INVENTION

Background—Through Scientific Research, and over 20+ years on the professional Golf Tours, working with over 300 Tour pros and over 3000 other golfers and coaches, SportPsych has found that the frequent winners on Tour (PGA, Sr. PGA & LPGA) measured differently from the other tour players on 8 of 32 Personality Traits. This research has provided Sportpsych with a unique means of developing training programs to assist professional and serious golfers on how to optimize their performance through mastering the mental techniques of the winning pros.

Purpose of the Invention—As part of the on-going development by Sportpsych to provide training and tools to the Pros, a novel product family, called the MindReader product line, has been developed. The purpose of the MindReader family of products is to assist in improving a player's mental game in competitive athletic events. Specifically, one of the 8 personality traits—relaxation/tension, is measured, and a unique index number, a performance predictor, if you will, is generated that a coach or player uses as feedback on his mental state. Then, using the mental relaxation techniques taught by SportPsych, in combination with the visual feedback of the performance predictor (arousal number), the player is then advised of his state while performing the physical event (ea. Swing the club). The player and coach quickly identify the optimum arousal numbers for good performances and can then practice to achieve them during every shot. The player can also use the invention to practice relaxing and quieting their mind separately from the performance with feedback of how well they are doing.

The Heart Parameter Monitor Invention: The Heart Rate Parameter invention (also referred to herein as the MindReader) is an electronic device designed to measure the level of mental activity (arousal) or relaxation of an individual. It does this through a precise physiological measurement of the interval between every heart beat. To this measurement is applied a proprietary algorithm which, through a series of mathematical calculations, computes a unique index number which is both a predictor of human performance, and a measure of mental activity or arousal and stress. There is substantial prior art and literature that demonstrates the high degree of correlation between mental activity levels, stress and heart rate variability. This invention takes the next step to produce a unique arousal or stress index number or performance predictor, that is valuable in the training and improving of one's mental game in any athletic event and in monitoring one's level of stress.

Apparatus: following is the hardware description and function.
The product hardware consists of three elements:

1. A Chest strap transmitter

2. The MindReader—There are two models, one for the player and one for a coach. Coach's model features a large display and is pager-sized, designed to clip to a belt or pocket where a coach can observe numbers at distance. It may be held in coach's hand near player. It can also connect to a computer or PDA for coaching with proprietary software. The Player's model is currently similar to the Coach's model in size and display but does not connect directly to a computer or PDA. The future planned Player embodiment is a wrist-worn form for individual usage. Player may use this alone, placing on the ground or hanging on belt where can he can see the display.

3. An optional ordinary personal computer or hand-held PDA, running a proprietary software application designed to provide breathing and relaxation training and, in the coaches version, so additional technical information, including a breathing metronome and graph chart of heart rate are provided.

Chest transmitter—The chest belt transmitter consists of sense electrodes placed on each side of the rib cage, just under the chest, and held in place with an elastic strap. State of the art devices today typically produce a 5KHz burst that lasts 20-25 milliseconds, upon each detected heart beat. The signal is transmitted magnetically because it is coupled to an oscillator consisting of a capacitor in parallel with an inductor, which are tuned to oscillate at 5KHz. A similarly fashioned LC oscillator is designed into the receiver for pickup. This system has severe range restrictions of 3-6 ft., and currently there is no provision for interference from one user to the next.

Therefore, it is contemplated to replace the magnetically coupled LC oscillators with modern, secure wireless technology, such as Bluetooth or 802.11xx (WiFi) to improve range, eliminate unit to unit interference, and provide security. This alternative wireless transmission does not otherwise alter the novelty of the invention.

PC or PDA software—Designed for use by coaches, PC software running in tandem with the MindReader, and during operation, produces additional visual feedback useful for training.

Mindreader Hardware—A justification of the key hardware choices is provided in the confidential memo attached) The core of the hardware consists of an 8-bit microcontroller with advanced power management, runnable on a lithium coin cell if necessary, along with the following necessary attributes:

ROM/RAM size of 32 k/512
Low active power 500 uA achievable
Usable low standby of 5-8 uA
UART or IRDA port
Other serial interface preferable SPI for serial memory
LCD controller
A/D 10 bit or higher
Pin count approx 48-52
Small package availability 10×10 or 12×12 mm sq.
Watchdog timer
Watch timer or means to implement accurate time keeping
1.8V to 3.3V operation
reasonable collection of timers/16-bit counters
Flash programmable for protos and early production

A good example is the NEC uPD78F9418, which was chosen for the preferred embodiment.

The hardware is a conventional implementation of a portable measuring instrument, tailored for this application, thus has little novelty on its own. The microcontroller is the heart of the circuit, and is required to do all the math calculations for the performance predictor, arousal or stress number. It is attached to a two-digit LCD, where the number is output. A small serial flash or EEPROM is attached for conventional serial storage of parameters and data.

An unregulated power supply consists of a 3 Volt battery, either a single lithium or a pair of alkaline. Diode D4 provides circuit protection in case the battery is installed backwards. A single op-Amp U6 provides the analog ground signal AGND, which is ½ of the battery voltage. The analog front end consists of a conventional 5KHz receiver, an R-wave detection circuit, and an RS-232 communications channel.

The 5KHz receiver reproduces the 20 ms long burst of 5 KHz signal produced by the chest transmitter, when the MindReader is close enough to the transmitter for the unit to magnetically couple to it. Inductor L1 and capacitor C3 form the tank oscillator circuit, signal of which is amplified first by Q1 then by Q2. The signal generated by Q2 is called 5 khz Burst, and is fed through a unity gain buffer U3A before going to the R-wave detector.

The R-wave detector consists of a full wave rectifier consisting conventionally of U3B, diode D2 and resistors R6 and R10. The output of this circuit is feed to an integrator, output of which rises in the presence of a 5 khz signal, stays high, and then goes low when the 5 kHz tone is gone. In affect, the output of this circuit, R14 and C11, provides an envelope signal around the 5 khz signal. This envelope signal is first sent to a peak detector to save its maximum height, and then to one input of the detection comparitor.

The peak detector consists of U3C, D3, C12, R12 and R13. This adaptive peak detector charges up C12 to 85% of the peak height of the envelope wave, and slowly bleeds down logarithmically at a 4.7 second time constant. This slowly declining threshold is necessary to adapt to varying signal sizes due to moving the MindReader away or towards the transmitter, and to adjust to noise bursts. When the output of the slowly declining peak detector meets the next envelope wave at the input of the comparitor, the comparitor fires, creating a digital pulse that is fed to the microcontroller interrupt pin, indicating the precise start of a heart beat.

### Software Algorithms and Processes

HRV Defined—Heart Rate variability is a statistical calculation defined as the standard deviation of a population of heart rate measurements. Its purpose is to provide a measurement of the amount of variation, or variability, in the sample set of heart rate measurements. Statistical variance is the usual calculation for this, and Standard Deviation (square root of variance) is the normalized, or standardized way of viewing the data sets. HRV has many uses, including prediction of heart attack and executive stress levels

Prior art and the literature, as well as SportPsych's own research, show that measuring Heart Rate Variability is a commonly applied, useful technique to measure tension/relaxation levels. Much literature exists to demonstrate the correlation between this physiological measurement, and mental state of activity or stress. Therefore the proprietary metric of the performance predictor (relaxation index) is derived and mathematically related to HRV.

One problem to solve was the following: the invention required that all calculations be done in the MindReader microcontroller, not in a PC, so that the MindReader could function as a stand-alone product. Yet the traditional methods of calculating HRV are computational intense for a simple 8-bit microcontroller, involving multiply, divide, square and square root operations with large floating point numbers. To do these calculations several times per second is not feasible while maintaining extremely low power operation. Therefore, a new method was needed that produced the equivalent information as HRV, yet was computationally achievable in real-time within the power budget of an 8-bit microcontroller. In addition, the data set had to be screened in real-time against a set of empirically determined physiological rules, subjected to a fast adjustment algorithm for rapidly transitioning data, and finally scaled non-linearly to fit the final data set into a numerical range of 0-99.

Operation proceeds as follows:
Upon detection of a heart beat (via hardware interrupt)

1. Precisely measure beat-to-beat interval in milliseconds.

2. Compare this measurement to physiological rule of +/−50% of the average

3. If ok, then proceed, else discard the interval as noise or an outlier. A background process tracks the number of discards, and corrects the flow if the count gets too high.

Note: Normal human beat-to-beat variation is no more than 10-15% according to the literature (will cite a reference if necessary)

However, empirical testing at SportPsych with subjects successful with mental relaxation techniques, demonstrate that their heart rates can vary more than 30%, much greater than the literature states. Therefore, this rule is proprietary.

A blanking period of 200 ms is applied and no other heartbeats can be detected. This is a physiological rule that says that no heart rates above 300 beats per minute are considered normal for the intended use of this instrument.

4. Once an interval is accepted as normal, it is placed in an 8 deep buffer, and an eight count moving average is calculated using integer arithmetic. Average heart rate can then be calculated by dividing 60000 by the average value of the buffer.

5. HRV equivalence calculation is then done according the following algorithm:

sum of [ABS(X−Xave)]/16 where X and Xave are the current and average millisecond interval measurements.

This simplified calculation substitutes well for square root of variance (standard deviation). In both methods, the difference between the current sample and the average is calculated. ABS (absolute value) takes the place of squaring, but achieves the necessary outcome of making each difference from the mean sample be a positive number. Yet computational efficiency is preserved without square root and squaring.

To make the HRV algorithm reactive to real-time heart rate variation, a depth of 16 difference variables was empirically determined to provide enough smoothing of the data, yet provide quick reaction to changes. The final HRV equivalent is thus determined as a 16 count moving average of the difference values (where difference means difference between the current measured interval and average of the previous 8).

At this point in the algorithm, a number is produced between 0 to approximately 125, with 0 meaning no beat to beat variation (such as a medical ECG simulator) and 125, which can be achieved by an aerobically fit person highly trained in SportPsych relaxation techniques, including respiration control.

6. With two digits allowed in the display, these numbers are then scaled to a range of 0-99. Any variety of scaling techniques can be used. A preferred method uses an integer divide by a power of 2, so computationally efficient shifts can be used instead of divide algorithms. An alternative is to calculate the 16-difference value average by dividing by 20, instead of 16, thus scaling the numbers 0-125 into a range of 0-99.

7. A background process determines if the person is “in the zone” and a flag is set. The flag is set if 4 of the most recent values are greater than 55.

If a the flag indicating in-zone is set, and suddenly the next four in row are lower, then a one time adjustment is made to the final output value. The final value is adjusted as follows:

new hrv value=hrv val/2+current difference val/4 but with the additional constraint that it cannot adjust to be smaller than the current difference.

This part of the algorithm has the net effect making a one time sudden change to the display value when the user has suddenly lost his mental training concentration, giving a rapid indication to the coach or the user.

During preliminary testing, it was determined that test subjects thought high numbers were “bad” and low numbers were “good”, just the opposite of the final relaxation number. Therefore, this final stage subtracts the current value from 100. This produces a final display value that yields high numbers when tense, or out of the training zone, and low numbers when in the zone, and relaxed or mentally quiet.

### Alternate Overview

This is a summary of my review of the major digital electronic components, microcontroller, LCD display, and memory, with selections based on tradeoffs required to meet certain product goals and attributes. Although this is a small prototype build, we must design it with production attributes to minimize design changes down the road, and to confirm component available and supply in the future. Therefore, we need these primary product attributes for the HRV monitor to be successful:

### Consumer Product Type of Cost Structure

Smallest reasonably priced components possible. (in other words, getting too aggressive on component size inflates cost beyond commodity pricing). We want all the electronics to fit in a belt-worn pager-sized package.

Lowest possible power to run the circuits up to one month on a single set of batteries. Secondary, but important considerations:

As part of the original product goals, we will avoid high custom chip NRE expense, and long development cycles. So we will limit device selections to off the shelf components, in standard packaging.

Component count to be minimized, software substituted wherever possible.

### The System Architecture

To achieve these goals, the electronics system minimally consists of these major elements: Battery and power supply regulation, microcontroller, memory, analog amplifiers and filters, LCD.

We need a small footprint microcontroller, preferably a 10×10 mm or 12×12 mm package. (Pin count drives size and cost of the micro). To achieve a small microcontroller footprint, we need to minimize the number of pins on the microcontroller. This in turn affects the overall system architecture.

For example, we need an off-chip memory device for the incoming ECG data stream, and variable storage arrays used for the HRV calculations. In a typical or classic memory interface, 32 pins are dedicated to address and data for the memory. Next largest user of pins is the LCD display, using up anywhere from 20 to 40 pins.

### LCD

By specifying a simple two digit display, (as opposed to using only 2 digits on a 3½ or 4 digit display) and using it in static or duplex mode, pins normally allocated to LCD can now be used for other functions, thus reducing overall pin count. Varitronix and others offer suitable off-the-shelf solutions.

### Analog

By using high integration operational amplifiers (4 per package), the need is eliminated for power and ground on 3 amplifiers for every 4 op-amps instantiated in the design. This is good savings in circuit board real estate, and reduced pin count. Selections are many, but one of the most suitable series of ultra-low power op-amps is offered by MAXIM, thus these will be used in the design.

### Memory

Due to the amount of data samples taken, and the data structures required for HRV calculations, a small memory chip is required. Focusing on the memory interface, I plan to use a new type of serial Flash memory, backed-up by the 1 k internal SRAM of the microcontroller. This new memory reduces memory to 3 pins from 32. (These memories are the similar to the ones used in cell phones and MP3 players, and digital cameras, thus are plentiful and inexpensive). In the HRV monitor, during operation, ECG sample data is stored in internal microcontroller SRAM, until reception of the next R-wave complex, when a new calculation of heart rate variability is performed. Once 128 byte page is accumulated, then it is programmed into a page of the serial RAM. This would occur about once per second. Once per second programming keeps the active power time of the Flash modulated to about 3 ms every 1.28 seconds (assuming 10 ms sample rate). Assuming active current is 15 ma, this averages to 35 uA during operation. Should this data one day need to be saved and played back (future feature) the memory would have the same 15 mA active current consumption, thus will require a similar power up-read-power down modulation technique during transmission to manage the overall power consumption.

Here is what we stand to gain with this new type of memory device:

1. PIn count to the memory is reduced from 32 to a 3 wire serial interface

2. Microcontroller pin count requirement reduces by about 28 pins

3. Board layout and routing simplifies—(potential cost reduction of circuit board)

These are the primary serial memory attributes the HRV monitor requires, along with some benefits:

1. Mbit in size—stores a running total of 15-20 minutes of ECG and HRV data

2. Bulk erase in less than 2 seconds, (reasonable time to avoid accidental erasure)

3. 128 byte page program in less than 3 ms (less than ½ ECG data sample time)

4. 2.7-3.3 V supply—one or two batteries

5. Active current less than 15 ma during record or playback—necessary to meet battery life goals

6. Low standby of 5 uA or less eliminates power control, or simplifies power management circuitry

7. 10,000 erase/program cycles (given multiple recordings and one transmission per day as worse case, this yields greater than 10 year product life. An extension can be created architecturally, by using on one 256 k sector (5 minutes of memory) until it has been cycled 10,000 times, and moving to the next sector for record space. Only sector erase (not bulk erase would be used in this extended life scenario) Another alternative is 100,000 cycle flash technology.

The ST Microelectronics M25P10 in a 150 mil package fits. An alternative is a 100,000 cycle device introduced last October by Silicon Storage Technology, Inc. The 1 mbit SST45VF010.

### Micro Solutions

Given a reduced pin count allowed by a serial memory device, a smaller foot print micro is now possible. It is presently preferred to use the NEC K0S micro family, specifically, the uPD789F417, as the micro of choice. The micros feature desired low power and have other attributes that make them effective.

### SUMMARY

A simplified memory-micro structure is possible using serial flash with a small footprint micro, cutting digital real estate in half. An ST M25P10 coupled to an NEC uPD 789417 or 789881. Coupled with a pair of Maxim quad op-amps, we have a complete electronics package.

### MindReader Family Technical Description

Backgound—Through Scientific Research, and over 20+years on the professional Golf Tours, working with over 300 Tour pros and over 3000 other golfers and coaches, SportPsych has found that the frequent winners on Tour (PGA, Sr. PGA & LPGA) measured differently from the other tour players on 8 of 32 Personality Traits. This research has provided Sportpsych with a unique means of developing training programs to assist professional and serious golfers on how to optimize their performance through mastering the mental techniques of the winning pros.

Purpose of the Invention—As part of the on-going development by Sportpsych to provide training and tools to the Pros, a novel product family, called the MindReader product line, has been developed. The purpose of the MindReader family of products is to assist in improving a player's mental game in competitive athletic events. Specifically, one of the 8 personality traits—relaxation/tension, is measured, and a unique index number, a performance predictor, if you will, is generated that a coach or player uses as feedback on his mental state. Then, using the mental relaxation techniques taught by SportPsych, in combination with the visual feedback of the performance predictor (arousal number), the player is then advised of his state while performing the physical event (ea. Swing the club). The player and coach quickly identify the optimum arousal numbers for good performances and can then practice to achieve them during every shot. The player can also use the invention to practice relaxing and quieting their mind separately from the performance with feedback of how well they are doing.

The MindReader Invention: The MindReader is an electronic device designed to measure the level of mental activity (arousal) or relaxation of an individual. It does this through a precise physiological measurement of the interval between every heart beat. To this measurement is applied a proprietary algorithm which, through a series of mathematical calculations, computes a unique index number which is both a predictor of human performance, and a measure of mental activity or arousal and stress. There is substantial prior art and literature that demonstrates the high degree of correlation between mental activity levels, stress and heart rate variability. This invention takes the next step to produce a unique arousal or stress index number or performance predictor, that is valuable in the training and improving of one's mental game in any athletic event and in monitoring one's level of stress.

Apparatus: following is the hardware description and function based on the schematics in the appendix.

The product hardware consists of three elements:

4. A Chest strap transmitter

5. The MindReader—There are two models, one for the player and one for a coach. Coach's model features a large display and is pager-sized, designed to clip to a belt or pocket where a coach can observe numbers at distance. It may be held in coach's hand near player. It can also connect to a computer or PDA for coaching with proprietary software. The Player's model is currently similar to the Coach's model in size and display but does not connect directly to a computer or PDA. The future planned Player embodiment is a wrist-worn form for individual usage. Player may use this alone, placing on the ground or hanging on belt where can he can see the display.

6. An optional ordinary personal computer or hand-held PDA, running a proprietary software application designed to provide breathing and relaxation training and, in the coaches version, so additional technical information, including a breathing metronome and graph chart of heart rate are provided.

Chest transmitter—The chest belt transmitter consists of sense electrodes placed on each side of the rib cage, just under the chest, and held in place with an elastic strap. State of the art devices today typically produce a 5KHz burst that lasts 20-25 milliseconds, upon each detected heart beat. The signal is transmitted magnetically because it is coupled to an oscillator consisting of a capacitor in parallel with an inductor, which are tuned to oscillate at 5KHz. A similarly fashioned LC oscillator is designed into the receiver for pickup. This system has severe range restrictions of 3-6 ft., and currently there is no provision for interference from one user to the next. Therefore, it is contemplated to replace the magnetically coupled LC oscillators with modern, secure wireless technology, such as Bluetooth or 802.11xx (WiFi) to improve range, eliminate unit to unit interference, and provide security. This alternative wireless transmission does not otherwise alter the novelty of the invention.

PC or PDA software—Designed for use by coaches, PC software running in tandem with the MindReader, and during operation, produces additional visual feedback useful for training.

Mindreader Hardware—A justification of the key hardware choices is provided in the confidential memo attached) The core of the hardware consists of an 8-bit microcontroller with advanced power management, runnable on a lithium coin cell if necessary, along with the following necessary attributes:

ROM/RAM size of 32 k/512
Low active power 500 uA achievable
Usable low standby of 5-8 uA
UART or IRDA port
Other serial interface preferable SPI for serial memory
LCD controller
A/D 10 bit or higher
Pin count approx 48-52
Small package availability 10×10 or 12×12 mm sq.
Watchdog timer
Watch timer or means to implement accurate time keeping
1.8V to 3.3V operation
reasonable collection of timers/16-bit counters
Flash programmable for protos and early production

A good example is the NEC uPD78F9418, which was chosen for the preferred embodiment.

The hardware is a conventional implementation of a portable measuring instrument, tailored for this application, thus has little novelty on its own. Referring to the schematic labeled GolfPsych HRV Digital Circuitry, the microcontroller is the heart of the circuit, and is required to do all the math calculations for the performance predictor, arousal or stress number. It is attached to a two-digit LCD, where the number is output. A small serial flash or EEPROM is attached for conventional serial storage of parameters and data.

An unregulated power supply consists of a 3 Volt battery, either a single lithium or a pair of alkaline. Diode D4 provides circuit protection in case the battery is installed backwards. A single op-Amp U6 provides the analog ground signal AGND, which is ½ of the battery voltage.

Refer to the schematic labeled Analog HRV Circuitry. The analog front end consists of a conventional 5KHz receiver, an R-wave detection circuit, and an RS-232 communications channel.

The 5KHz receiver reproduces the 20 ms long burst of 5 KHz signal produced by the chest transmitter, when the MindReader is close enough to the transmitter for the unit to magnetically couple to it. Inductor L1 and capacitor C3 form the tank oscillator circuit, signal of which is amplified first by Q1 then by Q2. The signal generated by Q2 is called 5 khz Burst, and is fed through a unity gain buffer U3A before going to the R-wave detector.

The R-wave detector consists of a full wave rectifier consisting conventionally of U3B, diode D2 and resistors R6 and R10. The output of this circuit is feed to an integrator, output of which rises in the presence of a 5 khz signal, stays high, and then goes low when the 5 kHz tone is gone. In affect, the output of this circuit, R14 and C11, provides an envelope signal around the 5 khz signal. This envelope signal is first sent to a peak detector to save its maximum height, and then to one input of the detection comparitor.

The peak detector consists of U3C, D3, C12, R12 and R13. This adaptive peak detector charges up C12 to 85% of the peak height of the envelope wave, and slowly bleeds down logarithmically at a 4.7 second time constant. This slowly declining threshold is necessary to adapt to varying signal sizes due to moving the MindReader away or towards the transmitter, and to adjust to noise bursts. When the output of the slowly declining peak detector meets the next envelope wave at the input of the comparitor, the comparitor fires, creating a digital pulse that is fed to the microcontroller interrupt pin, indicating the precise start of a heart beat.

### Software Algorithms and Processes

HRV Defined—Heart Rate variability is a statistical calculation defined as the standard deviation of a population of heart rate measurements. Its purpose is to provide a measurement of the amount of variation, or variability, in the sample set of heart rate measurements. Statistical variance is the usual calculation for this, and Standard Deviation (square root of variance) is the normalized, or standardized way of viewing the data sets. HRV has many uses, including prediction of heart attack and executive stress levels

Prior art and the literature, as well as SportPsych's own research, show that measuring Heart Rate Variability is a commonly applied, useful technique to measure tension/relaxation levels. Much literature exists to demonstrate the correlation between this physiological measurement, and mental state of activity or stress. Therefore the proprietary metric of the performance predictor (relaxation index) is derived and mathematically related to HRV.

The problem to solve was this: Our design required that all calculations be done in the MindReader microcontroller, not in a PC, so that the MindReader can function as a stand-alone product. Yet the traditional methods of calculating HRV are computational intense for a simple 8-bit microcontroller, involving multiply, divide, square and square root operations with large floating point numbers. To do these calculations several times per second is not feasible while maintaining extremely low power operation. Therefore, a new method was needed that produced the equivalent information as HRV, yet was computationally achievable in real-time within the power budget of an 8-bit microcontroller. In addition, the data set had to be screened in real-time against a set of empirically determined physiological rules, subjected to a fast adjustment algorithm for rapidly transitioning data, and finally scaled non-linearly to fit the final data set into a numerical range of 0-99.

Operation proceeds as follows:
Upon detection of a heart beat (via hardware interrupt)

6. Precisely measure beat-to-beat interval in milliseconds.

7. Compare this measurement to physiological rule of +/−50% of the average

8. If ok, then proceed, else discard the interval as noise or an outlier. A background process tracks the number of discards, and corrects the flow if the count gets too high.

Note: Normal human beat-to-beat variation is no more than 10-15% according to the literature (will cite a reference if necessary)

However, empirical testing at SportPsych with subjects successful with mental relaxation techniques, demonstrate that their heart rates can vary more than 30%, much greater than the literature states. Therefore, this rule is proprietary.

A blanking period of 200 ms is applied and no other heartbeats can be detected. This is a physiological rule that says that no heart rates above 300 beats per minute are considered normal for the intended use of this instrument.

9. Once an interval is accepted as normal, it is placed in an 8 deep buffer, and an eight count moving average is calculated using integer arithmetic. Average heart rate can then be calculated by dividing 60000 by the average value of the buffer.

10. HRV equivalence calculation is then done according the following algorithm:

sum of [ABS(X−Xave)]/16 where X and Xave are the current and average millisecond interval measurements.

This simplified calculation substitutes well for square root of variance (standard deviation). In both methods, the difference between the current sample and the average is calculated. ABS (absolute value) takes the place of squaring, but achieves the necessary outcome of making each difference from the mean sample be a positive number. Yet computational efficiency is preserved without square root and squaring.

To make the HRV algorithm reactive to real-time heart rate variation, a depth of 16 difference variables was empirically determined to provide enough smoothing of the data, yet provide quick reaction to changes. The final HRV equivalent is thus determined as a 16 count moving average of the difference values (where difference means difference between the current measured interval and average of the previous 8).

At this point in the algorithm, a number is produced between 0 to approximately 125, with 0 meaning no beat to beat variation (such as a medical ECG simulator) and 125, which can be achieved by an aerobically fit person highly trained in SportPsych relaxation techniques, including respiration control.

6. With two digits allowed in the display, these numbers are then scaled to a range of 0-99. Any variety of scaling techniques can be used. A preferred method uses an integer divide by a power of 2, so computationally efficient shifts can be used instead of divide algorithms. An alternative is to calculate the 16-difference value average by dividing by 20, instead of 16, thus scaling the numbers 0-125 into a range of 0-99.

7. A background process determines if the person is “in the zone” and a flag is set. The flag is set if 4 of the most recent values are greater than 55.

If a the flag indicating in-zone is set, and suddenly the next four in row are lower, then a one time adjustment is made to the final output value. The final value is adjusted as follows:

new hrv value=hrv val/2+current difference val/4 but with the additional constraint that it cannot adjust to be smaller than the current difference.

This part of the algorithm has the net effect making a one time sudden change to the display value when the user has suddenly lost his mental training concentration, giving a rapid indication to the coach or the user.

During preliminary testing, it was determined that test subjects thought high numbers were “bad” and low numbers were “good”, just the opposite of the final relaxation number. Therefore, this final stage subtracts the current value from 100. This produces a final display value that yields high numbers when tense, or out of the training zone, and low numbers when in the zone, and relaxed or mentally quiet.

We claim:

1. An electronic device to measure the level of mental activity of an individual, comprising:
measurement structure for measuring the interval between every heart beat of the individual;
analysis structure constructed to perform a series of mathematical calculations ultimately to compute an index number for the individual that is a measure of mental activity of the individual and is usable to predict the quality of the individual's performance in a desired activity.
